(19) United States
(12) Patent Application Publication
(10) Pub. No.: US 2023/0167400 A1
(43) Pub. Date: Jun. 1, 2023

(54) PRODUCTION OF FUNGAL BIOMASS FROM SIMPLE CARBON SOURCES

(71) Applicant: MYCOFOOD US LLC, London (GB)

(72) Inventors: Martin Blasco, Buenos Aires (AR); Horacio Claudio Acerbo, Buenos Aires (AR)

(73) Assignee: MYCOFOOD US LLC, London (GB)

(21) Appl. No.: 18/071,024

(22) Filed: Nov. 29, 2022

Related U.S. Application Data
(60) Provisional application No. 63/283,810, filed on Nov. 29, 2021.

(51) Int. Cl. C12N 1/14

(52) U.S. Cl. CPC C12N 1/14

(57) ABSTRACT

A modified Fusarium venenatum capable of metabolizing simple carbon sources is disclosed. Also provided are methods of producing biomass by administering a simple carbon source to the Fusarium veneatum, along with food compositions that include the produced biomass.

### CROSS REFERENCE TO RELATED APPLICATIONS

This application claims priority to U.S. Provisional Patent Application No. 63/283,810, filed Nov. 29, 2021, the contents of which are incorporated by reference in their entirety.

### FIELD OF INVENTION

The present invention is directed to the production of fungal biomass from simple carbon sources.

### SUMMARY

One general aspect of the invention includes a modified Fusarium venenatum capable of metabolizing simple carbon sources.

Another general aspect of the invention includes a method of producing a biomass by administering a simple carbon source to Fusarium venenatum.

Another aspect of the invention is the isolation of the biomass and use in a food composition.

### BRIEF DESCRIPTION OF THE DRAWINGS

FIG. 1 shows a scheme for carbon assimilation from acetate/ethanol in yeast via the glyoxylate cycle.

FIG. 2 shows a biomass production setup with a carbon dioxide-fixation step for carbon and energy source feeding.

### DETAILED DESCRIPTION

Embodiments of the invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

Unless otherwise indicated, all parts and percentages are by weight. As used herein, the term “about” refers to plus or minus 10% of the indicated value. Unless otherwise stated or made clear by context, weight percentages are provided based on the total amount of the composition in which they are described. As used herein, the singular forms “a,” “an,” and “the” include plural referents unless the context clearly dictates otherwise.

Use of biomass, for example biomass derived from fungi, to prepare a food composition has grown in popularity. Food products prepared from such biomass have been developed as sustainable food sources, for example as substitute for meat products. Use of biomass to prepare food compositions is thus known in the art, and new uses continue to be developed. However, there is a continuing need to develop methods of biomass productions that are sustainable or can be used in a range of environments.

Described herein are methods of producing fungal biomasses from simple carbon sources (i.e. C1-C2 carbon compounds). Simple carbon sources are herein defined as compounds containing 1-2 carbon atoms, one or more oxygen atoms, and hydrogen. Examples of simple carbon sources include, but are not limited to, formic acid, formaldehyde, methanol, acetic acid, acetaldehyde, ethanol, dimethyl ether, and salts thereof, for example, acetates and formates. The simple carbon sources used to produce the fungal biomass can derived from hydrocarbons, for example methane and ethane, which may be ultimately obtained from CO₂. In some embodiments, the simple carbon source acts as the energy source for the fungus.

The production of fungal biomass generally requires complex carbon sources, such as glucose, dextrose, starch, or cellulose, or other compounds or complex mixtures containing complex carbon sources, including without limitation agro-industrial waste products, such as molasses from sugar refining, vinasses from ethanol production, starchy material from potato processing facilities, or expellers from oil extraction from grains. There are no studies reporting the use of ethanol as a carbon source for biomass production; and while methanol is has been extensively used as a carbon source for yeast fungi, there are no reports of biomass production from filamentous fungi using methanol as a carbon source.

In the 1980's, Phillips Petroleum successfully produced a fungal biomass from the yeast species Pichia pastoris using methanol as a simple C1 carbon source. Despite this early success, its extension for the production of filamentous fungal biomass with simple carbon sources has not been further developed. This is likely due to the fact that fungi which can use simple carbon sources, including yeasts, generally yield very little biomass. For example, P. pastoris only yields 0.14 g of biomass per gram of methanol, compared to 0.6 g of biomass per gram of glycerol. There are currently no commercial fungal biomass products obtained from filamentous fungi that use simple carbon sources as their main production source. For example, Fusarium venenatum is a well-known biomass-producing filamentous fungus, but its biomass production relies on glucose as the carbon and energy source.

The yeast Schizosaccharomyces pombe consumes both acetate and glycerol, but metabolizes these different carbon sources via different metabolic pathways. Glycerol is directed towards glycolysis, gluconeogenesis, and the pentose phosphate pathway (PPP). In contrast, acetate is directed to the tricarboxylic acid cycle (TCA) for NADH production. Significantly, there currently is no available method of glycerol synthesis from carbon dioxide.

Obtaining microbial (e.g., fungal) biomass using carbon dioxide as the originating carbon source is considered an ideal solution for food production. However, carbon dioxide must be reduced to be a useable carbon source. The degree of reduction for organic compounds is generally defined as the number of equivalents of available electrons per carbon atom grams. For modelling purposes, the degree of reduction for microorganisms is the value used for the yield value calculations. The carbon and energy source required to obtain the biomass is calculated considering the degree of reduction of the biomass and the compounds involved in the production of the biomass. Whenever there is an imbalance between the energy required for the biomass production and the amount of the elements available for the biomass synthesis the yields would be affected. In those situations either the elements available or the energy would not be used efficiently. The generally observed yield with carbon sources with a degree of reduction exceeding that from biomass is 0.6 carbon mol in the biomass relative to mol of carbon in the substrate. The 0.6 yield is a desired value whenever the biomass is the product of interest. Given the degree of reduction of the biomass (generally slightly higher than 4), it is impossible to produce the biomass by direct use of carbon dioxide absent additional energy. A suitable solution for biomass production from heterotrophs with carbon dioxide is coupling the carbon dioxide fixation and reduction process to a microorganism capable of metabolizing the reduced fixation products, i.e. a reactor harboring microorganism. This could also be used to simultaneously resolve issues related to carbon dioxide buildup in enclosed systems, by allowing for the capture of carbon dioxide from carbon oxidation processes, such as respiration and/or combustion.

One such carbon dioxide reduction process is Sabatier's reaction is shown in Scheme 1 (Junaedi et al., 2011):

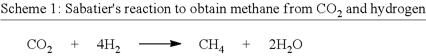

Sabatier's reaction is used for water recycling operations in spaceships where excess carbon is an effluent that is fully recycled by its conversion to readily consumable carbon sources for microorganisms. Adjusting Sabatier's electrochemical and biochemical reaction conditions allows for the conversion of carbon dioxide into reduced simple carbon sources which can be metabolized by microorganisms for growth. Depending on the reduction reaction conditions employed, various simple carbon sources can be obtained, including formic acid, formaldehyde, methanol, acetic acid, acetaldehyde, ethanol, and salts thereof, for example, formates and acetates among others. For example, performing Sabatier's reaction on nanodiamond results in the production of acetic acid, as shown in Scheme 2 below (Liu et al., 2015):

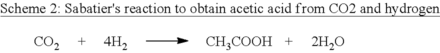

Employing these simple carbon sources results in biomass production that is uncoupled from photoautotrophic organisms, and has the potential to turn biomass production beginning with carbon dioxide into a more energy-efficient process, although energy may still be required to transform carbon dioxide into a suitable energy source.

Acetate, a C2 simple carbon source, can be produced from the electrocatalytic reduction of an aqueous carbon dioxide solution as disclosed in reports by Liu et al. in 2015, Zha et al. in 2020, and Zhang et al. in 2021. The processes are extremely efficient, with faradaic efficiencies reported as high as 96.5%, suggesting that the production of biomass via carbon dioxide reduction is an achievable goal if acetate can be used as a carbon source.

Acetogenesis is the biochemical process used by anaerobic bacteria to fix carbon dioxide in the presence of hydrogen to produce acetate. However, even when bacteria are able to produce biomass directly from acetate, the biomass produced lacks texture and is poor in quality, making it an unsuitable food source. One potential method for resolving this issue is feeding a solution of acetate to a microorganism suitable for human consumption whose metabolism is coupled to acetate consumption. This process can then be selectively directed to produce high value biomass products which can be used as food.

An additional concern is that acetate can also play a contradictory role in bacterial metabolism. For example, E. coli produces acetate by overflow metabolism when grown in glucose, which leads to growth inhibition without affecting central metabolism (Pinhall et al., 2019). Also, in yeast, acetate plays a role in triggering programmed cell death, making it an unsuitable carbon source for yeast-based biomass production (Chaves et al., 2021).

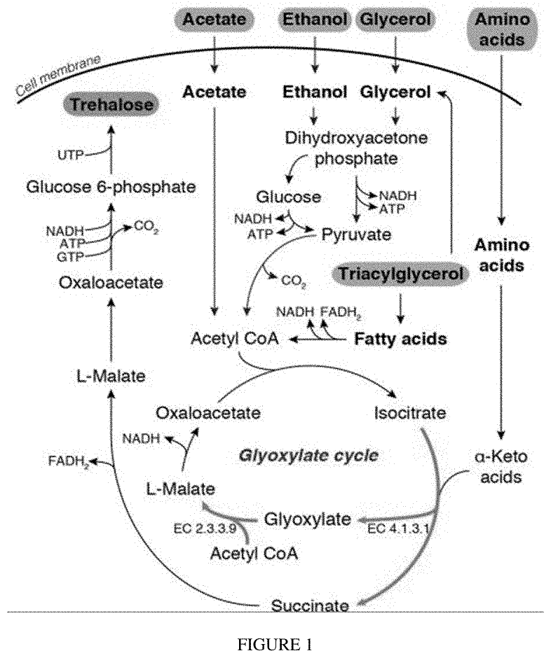

Despite 30 years of use in food production, currently there are no reports of biomass production by Fusarium venenatum using simple C1 or C2 carbon sources. Malate synthase and isocitrate lyase are two key enzymes involved in the glyoxylate cycle, which is responsible for the assimilation of acetate while bypassing the two decarboxylation steps observed in the tricarboxylic acid cycle (TCA) (Kornberg 1996). Fusarium venenatum possesses these two key enzymes in its genome, which make it a viable option for biomass production wherein acetate is employed as the carbon source according to the mechanism shown in FIG. 1, which shows a scheme for carbon assimilation from acetate/ethanol in yeast via the glyoxylate cycle

As shown in Scheme 3, isocitrate lyase (EC: 4.1.3.1) catalyzes the reaction of isocitrate (C00311) to produce succinate (C00042) and glyoxylate (C00048), as shown in Scheme 3 below:

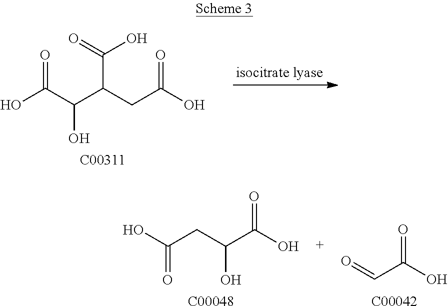

Malate synthase (EC: 2.3.3.9) then catalyzes the reaction of acetyl-CoA (C00024) with glyoxylate (C00048) to obtain malate (C00149), as shown in Scheme 4 (C00010 represents coenzyme A).

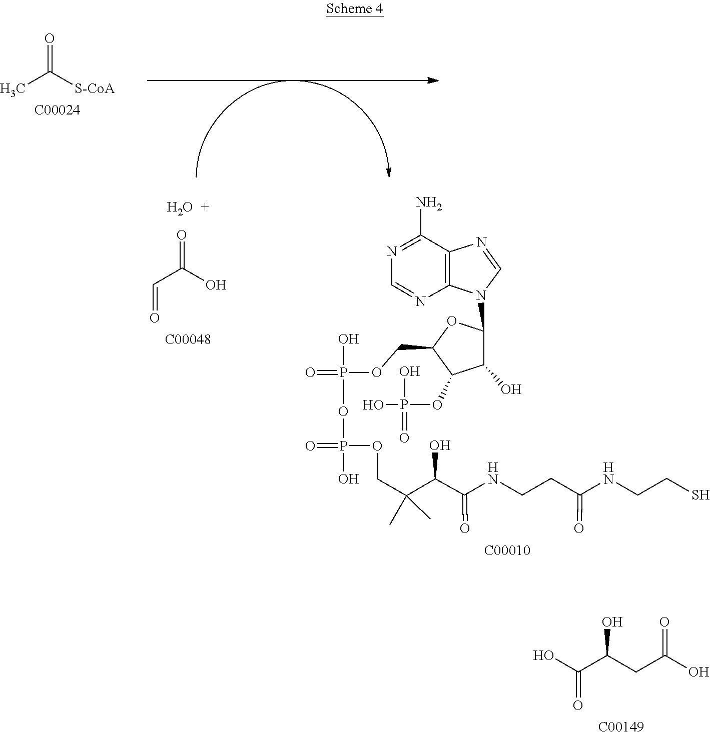

The malate is then further metabolized by the microorganism, leading to the building blocks for biomass production.

Coupling the carbon dioxide fixation occurring in an external source with microbial growth within a bioreactor allows for biomass production from filamentous fungi without the need for complex carbon sources. The use of a selection mechanism and a proper engineering of fungal strains capable of metabolizing acetate and methanol at faster rates than the wild type strains is the key to unlock the potential for utilizing simple carbon sources for efficient fungal biomass production for food.

Herein described is a method for food production from fungi without the need for autotrophic organisms coupled to its production. The method involves coupling carbon dioxide reduction together with a modified or engineered strain of Fusarium venenatum which efficiently produces biomass from carbon dioxide reduction products, such as acetate. Biomass from the Fusarium can then be utilized in food production processes as known in the art. The process resolves many drawbacks related to biomass production in harsh environmental conditions or situations where certain carbon sources are inaccessible, or when carbon recycling is required, such as in outer space.

### Example 1: Improvement of Biomass Production from Methanol by Metabolic Engineering

Low biomass production observed with moderate concentrations of methanol (4 g/L) is indicative of stressful conditions for fungal development that are not resolved by successive culture adaptation steps, suggesting a biochemical impairment for methanol metabolism. Considering the methylotrophs metabolic pathway has a high efficiency for methanol assimilation, an engineered Fusarium venenatum strain was designed to improve the metabolism of methanol by modifying key aspects of the peroxisome biochemistry. These modifications allow for the integration of the microorganism to carbon dioxide reduction devices for the production of biomass, presenting a promising solution for food production from methanol.

In the most conservative approach, the alcohol oxidase gene (i.e. AOX; GeneBank Accession number: KAG8360490) of Fusarium venenatum is duplicated and modified by adding a carboxy terminal signaling peptide for peroxisome targeting, similar to that of the efficient methylotrophic yeast, Pichia pastoris (also known as Komagataella phaffi), observed by Waterham et al. in 1997. The peptide used for this purpose is the Fusarium spp. peroxisome targeting signaling tripeptide disclosed by Moonil Son et al. in 2013. This presence of the tripeptide results in an increase in the titer of AOX for alcohol metabolism, while also relocating the enzyme from the cytosol to the peroxisome for compartmentalization of the metabolism, thereby ensuring microorganism cell viability. This specialized location is able to accommodate and contain the reactive oxidative species produced by formaldehyde synthesis as shown in Scheme 5 below:

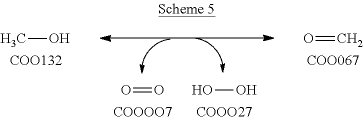

Methanol (C00132) is oxidized to formaldehyde (C00067) by AOX (EC: 1.1.3.13), which also results in the production of hydrogen peroxide (C00027). After methanol metabolism is achieved by AOX, formaldehyde production is coupled to peroxide reduction by a Komagataella phaffii catalase (GeneBank Accession Number XM_002492030) expression with the peroxisome targeting single 1 variant (PTS-1; SKL) at the c-terminal end. The Komagataella phaffii catalase (EC 1.11.1.6) reacts in the peroxisome for hydrogen peroxide (C00027) detoxification, resulting in water (C0001) and oxygen (C0007) as shown in Scheme 6 below.

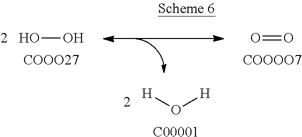

This reactive oxygen species scavenging system within the peroxisome consists of various additional species involved in controlling the presences of reactive species, including, for example, catalase (CAT), superoxide dismutase (SOD1), peroxiredoxin 5 (PRDX5), glutathione S-transferase kappa (GSTK1), ‘microsomal’ glutathione S-transferase (MGST1), epoxide hydrolase 2 (EPHX2), reduced glutathione (GSH), and vitamin C (VitC). The combined expression of this enzyme set, AOX, catalase, and the extra dihydroxyacetone synthase from Komagataella phaffii (GeneBank Accession Number FJ752552) within the peroxisome yields a several fold increase in biomass yield from methanol, thereby subverting the low yield and growth rate observed at low methanol levels with the wild type strains.

The strategy for incorporating the genes within the fungal genome involves transformation of the fungi with a strategy directed towards homologous end joining recombination to avoid non-controlled integration in the genome. The appropriate modulation of enzyme production is achieved by using a library with random variants of promoters involved in the expression of key metabolic pathway related enzymes (GeneBank Accession Number XP_025590498.1, XP_011320034), allowing a combinatory approach followed by continuous culture directed selection to ensure the enrichment of those strains with a greater ability for growth in methanol. Strains selected by this approach are banked and fully sequenced to ensure the genetic background and modifications as compared to the wild type strain.

### Example 2: Biomass Production from Acetate by Selective Adaptation

Spore germination and mycelium development is completely inhibited in saline medium containing acetate at a concentration of 67 mM (equivalent to 4 g/L of acetic acid), thereby precluding biomass production even at moderate levels of acetate. The inhibitory effect of acetate on yeast cell growth is even stronger, with inhibitory levels reported to be as low as 10 mM (Sousa et al., 2012). For fungi, the widespread inhibitory effect of acetate is attributed to several pathways, including i) the sensitivity of kinases to low acetate concentrations, which triggers programmed cell death pathways (PCDP), ii) the energetic burden of deprotonation upon entry into the cytosol which requires proton pumping to keep the internal pH within physiologically acceptable levels, and iii) failure to metabolize cellular building blocks due to a lack of efficient metabolic pathways.

Several approaches have been devised to overcome the problems related to acetate utilization by Fusarium venenatum. For example, modification of the pH of the culture medium avoids the direct transport of acetic acid but not acetate, thereby eliminating the proton balancing requirement on acetate entry. However, poor growth rates due to low substrate feeding is common, yielding low biomass production, thereby requiring larger equipment and higher costs.

In order to overcome the problems associated with acetate, acetate-induced death-resistant development of fungal strains is paramount. Given the moderate growth rate of Fusarium venenatum, and the potential to selectively enrich the acetate-induced death resistant population using increasing acetate concentrations in a continuous culture mode, this approach is combined with a synthetic biology approach directed toward generating a fast acetate growing strain.

The selective enrichment of acetate-resistant microorganism is performed by growing the strain subjected to selection in a batch culture, followed by feeding of acetate at increasing dilution rates (D). During the process, biomass production is continuously monitored to ensure constant biomass levels within the reactor. The base dilution is 0.05/hour and the rate increases as a function of time (t) according to the equation below:

D(t)=(3.3984×10⁻⁵×t²)−(9.2411×10⁻⁴×t)+564.65

The synthetic biology approach involves transformation of the strain with a combinatory library of promoter enzymes and transporters to ensure proper growth at high growth rates.

The strains retained within the reactor are selected based on their morphology by filtration and isolation for later characterization by genome sequencing. The combination of promoter-ORF is defined and the strains are reconstituted by transformation with the particular promoter ORF combination to recover the strain and validation. The enzymes selected encompass all those discussed above, and other proteins whose expression or repression is used to improve the protein production, including, but not limited to, calnexin (C1xA), immunoglobulin binding protein (BiP), protein disulfide isomerase (PDI), the GTPase (nucleotide guanosine triphosphate hydrolase) RacA and their combinatorial versions obtained by chemical synthesis. The best combination is selected by using an increasing dilution rate in a continuous culture process.

Considering that the metabolic pathway of acetate assimilation in Fusarium does not differ from that observed in other fungi with well-documented growth in acetate, it is suggested that most of the growth inhibition has a regulatory origin rather than metabolic origin. The suggested approach above could result in the improvement of the regulatory as well as metabolic aspects of this fungal species.

### Example 3: Growing Fusarium venenatum Using Simple Carbon Sources

Fusarium venenatum cells are generally grown from macroconidia in an aerobic bioreactor with a saline culture medium. An exemplary culture medium composition is shown in Table 1 below. In the production process, the addition of biotin at microgram level accelerates the reaction onset around the inoculation time. Thereafter, the biotin requirement is self-sustained.

TABLE 1
Culture medium composition for growing F. venenatum biomass

| Compound | Concentration (g/L) |
|---|---|
| K₂SO₄ | 1 |
| H₃PO₄ | 0.75 |
| MgSO4*7H₂O | 0.6 |
| CaCl₂ | 0.3 |
| ZnSO₄*7H₂O | 0.05 |
| MnSO₄*4H₂O | 0.02 |
| FeSO₄*7H₂O | 0.001 |
| CuSO₄*5H₂O | 0.0025 |

The culture medium composition of Table 1 can support the production of 20 g/L of fungal biomass provided enough of carbon and energy source is fed.

The carbon and energy source is added in the batch, is batch fed, or continuously fed at a non-toxic concentration. For fed batch or continuous feeding, growth is kept at a pseudo-steady/steady state, ensuring that the concentration of the carbon source is below the inhibitory concentration, which for acetate typically means a concentration of less than 40 mM. In addition to the carbon and energy source, the culture is accompanied by the addition of a nitrogen source, such as ammonia, to ensure a carbon to nitrogen ratio of about 1:0.2. The ratio is optimized to account for carbon incorporation into the biomass. Ammonia is the main nitrogen source for Fusarium under these culture conditions and is required to be present in the culture medium to control pH value at 5.8, the optimal for this process.

The carbon and energy source can be added from pure sources, or can be obtained from the reduction of carbon dioxide via equipment coupled to the bioreactor. When carbon dioxide reduction equipment is utilized, a filter is employed to ensure purity of the material within the bioreactor and avoid the migration of salts from the reduction equipment into the reactor. By using a hydrophobic membrane, carbon sources, such as acetate or ethanol, can be transferred into the bioreactor. In the case of acetate production systems, a low pH (below 3) is used to ensure its protonation and diffusion through hydrophobic membrane materials. This same strategy can be employed for ethanol-based production systems but without the requirement to lower the pH to allow the diffusion of ethanol in hydrophobic membranes.

Diffusion based devices such as membranes can also be used to deliver other components with high diffusion coefficients, thereby allowing their supply whenever the mass transfer is enough to supply the biomass with the nutrients available at the transfer interface. For example, another key component to produce biomass is nitrogen, which is frequently supplied as ammonia. Ammonia is a small molecule that diffuses through hydrophobic media when in aqueous solutions with a pH above 8. While this supply method has been used to deliver oxygen to low oxygen demanding culture mediums, it has not been adapted previously for the bioprocesses disclosed herein. Ammonia has a permeability to rubber of between 60-120 L/m²/day, whereas that of oxygen is 4.8-5.3 L/m²/day. Data for acetic acid is not available, except for pervaporation devices used for acetic acid purification (Tejraj & Udaya, 2012), which are also viable devices for biomass processing. By exploiting differences in the diffusion properties of various nutrients, the potential for contamination of the bioreactor is significantly reduced.

The feed ratio is regulated according to the oxygen consumption of the microorganism to ensure a fully aerobic environment for the fungi, while the carbon source is obtained from other sources and fed into the reaction as is or is combined with other nutrients to ensure an optimal balance of the components. Additionally, spent culture medium can be processed and recycled into the reactor, for example by the replenishment of spent components, and the carbon source is replenished via different transfer mechanisms, such as membrane diffusion, direct addition, or chemical transformation.

The carbon and energy source addition rate are adjusted to ensure the concentration is below the inhibitory level to avoid toxicity of the substrate to the microorganism in order to avoid low productivity and impaired biomass yield.

Cultivation under the optimized conditions described above typically results in the production of 0.5-0.6 g of biomass per gram of substrate (i.e. glucose or another carbon/energy source), which is near the practical maximum for biomass production on substrates with a degree of reduction that is slightly higher than the biomass.

An exemplary production setup is shown in FIG. 1. Therein, the culture medium preparation includes a device capable of producing carbon dioxide-derived reduction products which are metabolized by the fungal species which has been adapted or modified as discussed below.

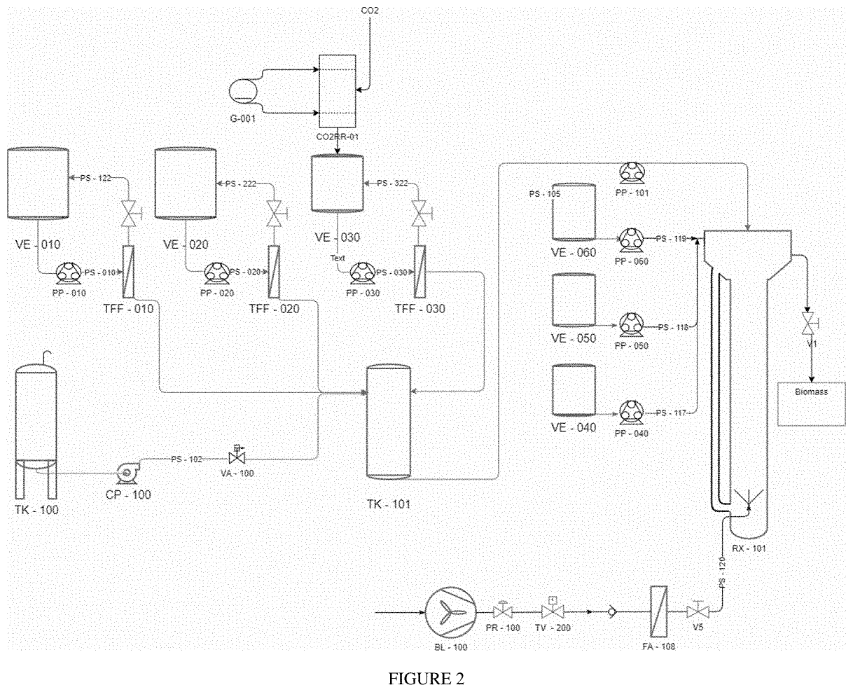

FIG. 2 shows a biomass production setup with a carbon dioxide-fixation step for carbon and energy source feeding. In the flow diagram, CO2RR-01 is an electrocatalytic reactor capable of reducing carbon dioxide to methanol, ethanol, or acetate in the presence of an aqueous solution of electrolytes. CO2RR-01 is connected to an electrical generator, G-001. The methanol, ethanol, or acetate is collected in VE-030, from which it is then transferred to the culture medium preparation tank, TK-101, and combined with other nutrients from tanks VE-010 and VE-020. VE-010 contains K₂SO₄, H₃PO₄, Mg²⁺, Zn²⁺, Mn²⁺, salts, and biotin; VE-020 contains a Fe′ salt solution; and TK-100 contains water. All of these tanks feed into the bioreactor, RX-101. Other components can be added as needed to provide the appropriate culture conditions. For example, in the system of FIG. 2, VE-040 contains an antifoam agent to avoid excess foam build up on the surface of the liquid within the bioreactor; VE-050 contains an acidic component to control pH; and VE-060 contains ammonium hydroxide to control pH and to provide nitrogen to the culture. Components designated TFF are membranes which are used for tangential flow filtration. BL-100 is compressed air for respiratory functions of the cells grown within the bioreactor.

The fungi within the reactor grows by consuming nutrients and oxygen to produce biomass. The biomass is then harvested as a wet solid and the culture supernatant is recycled as an aqueous solution to prepare a new culture medium. Any materials remaining after harvesting of the biomass are sterilized and incorporated into the new culture medium to maximize the biomass yield based on carbon. As an added bonus, any carbon dioxide generated from the bioreactor (typically present at a 5% concentration in the air) is reduced by the carbon dioxide reduction device to provide additional carbon and energy.

The production process can be further improved by inducing the initial fungal growth with complex carbon sources, and further improved by adding biomass heat treated extracts obtained during pasteurization of the biomass. The heat-treated extracts contain highly valuable nutrients for fungal growth which can be optionally fed back into the reactor, for example, via a tank similar to VE-010 shown in FIG. 2. Once the biomass is built up in the bioreactor, the culture medium is gradually ramped up to a full-acetate composition to ensure adaptation of the microorganism to the culture conditions.

When using the regular wild type strains of Fusarium venenatum for biomass production with methanol (about 0.4%) as the simple carbon source, the yields obtained were about 5% of those experienced when using glucose as the carbon source, indicating a drawback in carbon metabolism. Similar results were obtained when ethanol was employed in the culture medium. However, when acetate was used as the carbon and energy source no biomass production was observed, indicating wild type strains of F. venenatum are not viable for biomass production from acetate.

However, it was hypothesized that the fungi could be modified in order to allow for the metabolism of the simplest reduced carbon dioxide reduced compound, methanol. A flexible metabolism for biomass growth was devised by intervening in the metabolic pathways involving the anaplerotic steps in the TCA, glyoxylate cycle, and the adjustment of cytosolic reactions and placement of transporters. To this end, the metabolic pathway for methanol metabolism in the peroxisome was reconstructed by the expression and/or targeting of key enzymes.

Further aspects of the present disclosure are provided by the subject matter of the following clauses.

A modified Fusarium venenatum capable of metabolizing simple carbon sources.

The modified Fusarium venenatum of the previous clause produced by genetic modification. The modified Fusarium venenatum of the previous clause produced by adaptation.

A method of producing a biomass comprising administering a simple carbon source to Fusarium venenatum.

The method of any previous clause, wherein the simple carbon source is selected from the group consisting of formic acid, formaldehyde, methanol, acetic acid, acetaldehyde, ethanol, dimethyl ether, and salts thereof.

The method of any previous clause, wherein the simple carbon source is methanol or acetate.

The method of any previous clause, wherein the simple carbon source is methanol.

The method of any previous clause, wherein the simple carbon source is acetate.

The method of any previous clause, wherein the Fusarium venenatum is a modified Fusarium venenatum.

The method of any previous clause, where the modified Fusarium venenatum is produced by genetic modification.

The method of any previous clause, where the modified Fusarium venenatum is produced by adaptation.

The method of any previous clause, further comprising administering a nitrogen source to the Fusarium venenatum.

The method of any previous clause, wherein the concentration of the carbon source is maintained below an inhibitory concentration.

The method of any previous clause, wherein the simple carbon source is generated by reduction of carbon dioxide.

The method of any previous clause, wherein the reduction of carbon dioxide is performed by a biochemical reaction.

The method of any previous clause, wherein the reduction of carbon dioxide is performed by an electrocatalytic reaction.

The method of any previous clause, wherein the reduction of carbon dioxide is performed by Sabatier's catalytic dependent reaction.

The method of any previous clause, further comprising isolating the biomass.

A biomass produced by the method of any previous clause.

A food composition comprising the biomass of produced by the method of any previous clause.

The various embodiments described above are provided by way of illustration only and should not be construed to limit the scope of the disclosure. Various modifications and changes may be made to the principles described herein without following the example embodiments and applications illustrated and described herein, and without departing from the spirit and scope of the disclosure.

1. A modified Fusarium venenatum capable of metabolizing simple carbon sources.

2. The modified Fusarium venenatum of claim 1, produced by genetic modification.

3. The modified Fusarium venenatum of claim 1, produced by adaptation.

4. A method of producing a biomass comprising administering a simple carbon source to Fusarium venenatum.

5. The method of claim 4, wherein the simple carbon source is selected from the group consisting of formic acid, formaldehyde, methanol, acetic acid, acetaldehyde, ethanol, dimethyl ether, and salts thereof.

6. The method of claim 4, wherein the simple carbon source is methanol or acetate.

7. The method of claim 4, wherein the simple carbon source is methanol.

8. The method of claim 4, wherein the simple carbon source is acetate.

9. The method of claim 4, wherein the Fusarium venenatum is a modified Fusarium venenatum.

10. The method of claim 9, wherein the modified Fusarium venenatum is produced by genetic modification.

11. The method of claim 9, wherein the modified Fusarium venenatum is produced by adaptation.

12. The method of claim 4, further comprising administering a nitrogen source to the Fusarium venenatum.

13. The method of claim 4, wherein the concentration of the carbon source is maintained below an inhibitory concentration.

14. The method of claim 4, wherein the simple carbon source is generated by reduction of carbon dioxide.

15. The method of claim 14, wherein the reduction of carbon dioxide is performed by a biochemical reaction.

16. The method of claim 14, wherein the reduction of carbon dioxide is performed by an electrocatalytic reaction.

17. The method of claim 14, wherein the reduction of carbon dioxide is performed by Sabatier's catalytic dependent reaction.

18. The method of claim 4, further comprising isolating the biomass.

19. A biomass produced by the method of claim 18.

20. A food composition comprising the biomass of claim 19.
